(19) United States
(12) Patent Application Publication
(10) Pub. No.: US 2024/0076605 A1
(43) Pub. Date: Mar. 7, 2024

(54) METHOD IN BIOPROCESS PURIFICATION SYSTEM

(71) Applicant: CYTIVA SWEDEN AB, Uppsala (SE)

(72) Inventors: ANDREAS AXÉN, UPPSALA (SE); HELENA VESTERBERG, UPPSALA (SE); MAGNUS WETTERHALL, UPPSALA (SE); CAMILLA ESTMER NILSSON, UPPSALA (SE)

(73) Assignee: CYTIVA SWEDEN AB, UPPSALA (SE)

(21) Appl. No.: 18/272,249

(22) PCT Filed: Feb. 1, 2022
(86) PCT No.: PCT/EP2022/052298

§ 371 Date: Jul. 13, 2023

(30) Foreign Application Priority Data
Feb. 9, 2021 (GB) 2101775.1

(51) Int. Cl. C12M 1/36; C12M 1/00; C12M 1/34

(52) U.S. Cl. CPC C12M 41/48; C12M 29/10; C12M 29/24; C12M 41/12; C12M 41/26; C12M 41/30; C12M 41/46; C12M 47/10

(57) ABSTRACT

The present invention relates to a method for controlling process parameters in a bioreactor when producing a target product with a pre-determined critical to quality, CTQ, profile. The method comprises: monitoring (S20) at least one product quality attribute, PQA, for the target product; identifying (S30) trends and/or deviations by comparing the monitored at least one PQA to the CTQ profile of the target product; and controlling (S40) process parameters of the bioreactor based on the identified trends and/or deviations to maintain the target product within the CTQ profile.

### TECHNICAL FIELD

The present invention relates to a method for monitoring product quality parameters and controlling process parameters to obtain a target product from a bioreactor system.

### BACKGROUND

The quality of the material produced in the bioreactor (in cell culture system) is important to monitor in order to achieve a reliable, robust and economic manufacturing procedure and producing a safe product.

Therapeutic and other proteins generated by cells expressing proteins in a bioreactor are commonly defined regarding suitability for their purpose by a number of critical Product Quality Attributes, PQAs. These attributes can be polypeptide sequence variations, posttranslational modifications or other variants caused by e.g. enzymatic, chemical or thermal influence.

Commonly, the PQA are analyzed in an off-line setting, e.g. in an analytical laboratory, based on samples taken from the bioreactor at various points of time. Based on the collected conditions, such as pH, temperature, stirring rate, Dissolved Oxygen (DO), partial pressure of carbon dioxide (pCO2), and nutrients in the bioreactor, are altered in a way that makes the generation of the product with a desired critical-to-quality, CTQ, profile as favorable as possible.

The optimization of the conditions is commonly made based on experience and model-building from a large number of experiments for the specific process to be optimized.

A drawback is that the collection of data relating to the variations in PQA of the target product over the course of a full cultivation cycle is labor intensive and cumbersome. Additionally, many of the PQA of the target product are not obtain within the time course of the production process, but days after the production is finished.

Thus, there is a need to modify both the collection of data regarding PQA as well as the control process of the bioreactor to achieve a more efficient process to produce a target product.

### SUMMARY

An object of the present disclosure is to provide methods and devices configured to execute methods and computer programs which seek to mitigate, alleviate, or eliminate one or more of the above-identified deficiencies in the art and disadvantages singly or in any combination.

The object is achieved by a method as defined by the independent claims.

An advantage is that the quality of the target product is the input for control of the bioreactor allowing for a target product with a more narrow variations of PQA within a Critical-To-Quality, CTQ, profile of the target product.

An advantage with one embodiment is that when measuring Product Quality Attributes, PQAs, in real time (or near real time) other data in soft sensing and self-learning systems may be used to further improve the control of process parameters.

Further objects and advantages may be obtained from the detailed description by a skilled person in the art.

### BRIEF DESCRIPTION OF THE DRAWINGS

FIG. 1 illustrates an overview of a bioprocess purification system designed to purify a target product from a harvest fluid from a bioreactor.

FIG. 2 illustrates a prior art cell culture system.

FIG. 3 illustrates a schematic picture of a control system for an example embodiment of a cell culture system.

FIG. 4 is an illustrative example of a control unit.

FIG. 5 illustrates an example embodiment of a process for monitoring and controlling process parameters in a cell culture system.

FIGS. 6a-6e illustrate graphs exemplifying the behavior of selected PQAs in a cell culture process without implementing the process described in relation to FIG. 5.

FIG. 7 is a graph illustrating monitored mAb N-glycosylation profile in a bioreactor as a function of cell culture day.

FIGS. 8a-8c illustrate graphs used to control selected PQA.

### DETAILED DESCRIPTION

A bioprocess purification system is designed for production and purification of target products (such as proteins, biomolecules from cell culture/fermentation, natural extracts) by growing cells capable of expressing the target product in a cell culture bioreactor followed by a downstream purification process (also referred to as Downstream process) for purifying the target product. The downstream purification process may be any suitable process capable of providing a purified target product, the process may comprise one or multiple steps. One commonly used step in a downstream purification process is chromatography. In particular the current invention relates to a bioprocess purification system arranged to produce and provide a purified target product during a period of time where the target product is harvested from the bioreactor and purified by the downstream purification process while the cell culture is maintained. This includes different types of cell culture processes, such as a batch type cell culture process or a continuous cell culture process. Examples of continuous cell culture processes include perfusion cell culture and chemostat cell culture.

The downstream purification process is intimately connected to the quality of the target product during production. The main focus of this application is to improve the quality of the target product in the harvest intended to be introduced into a purification process, continuous or batch process, by ensuring that attributes critical to the quality of the target product are monitored and controlled during the production process.

In the description, two essential expressions are used to illustrate the inventive concept: PQA and CTQ profile. These expressions are related to each other and the quality of the target product in the feed from the cell cultivation bioreactor.

Definition of Product Quality Attribute, PQA

PQA is a measurable property that is important for the quality of the target product, and the PQA may be determined based on acquired data from sensors within the system and/or external equipment. Every PQA may be represented as a certain value that should be within a pre-determined interval.

Definition of Critical-to-Quality, CTQ, Profile

CTQ profile of a target product represents a set of intervals for different PQAs.

Illustrative Example

In this example, a number of PQAs have been identified as critical for the quality of a target product. The different PQAs are illustrated as PQA-1, PQA-2 and PQA-3 in this example.
CTQ profile of target product
PQA-1: 0.3-0.6 (set point 0.45)
PQA-2: 34-45 (set point 39.5)
PQA-3: 1-1.7 (set point 1.35)

The different PQAs are monitored over time based on data acquired from system sensors, such as temp sensors, pH sensors, and/or external equipment, such as Mass Spectrometer, as illustrates in table 1.

TABLE 1

| Time | PQA-1 | PQA-2 | PQA-3 |
|---|---|---|---|
| t1 | 0.53 | 40 | 1.25 |
| t2 | 0.55 | 42 | 1.4 |
| t3 | 0.52 | 44 | 1.3 |
| Average value over time | 0.533 | 42 | 1.317 |

Based on these values a trend analysis may be performed revealing that PQA-2 is on its way out of the interval defined by the CTQ profile and appropriate measures are needed to reduce PQA-2 by adjusting e.g. pH, temperature, stirring rate, DO, pCO2, or nutrients in the bioreactor nutriments without causing PQA-1 and PQA-3 to end up outside their respective interval.

Alternatively, the deviations from the set point for each PQA may be monitored to identify available options to improve the bioreactor process. E.g. the average value of PQA-1 over time is 0.533 which is 18.5% higher than the PQA-1 set point value of 0.45. This may be used as an indication to adjust process parameters in the bioreactor.

In some embodiment, a specific PQA may be considered to be more important than another PQA in the CTQ profile. In order for this to be reflected when determining available options to adjust process parameters, different weights may be assigned to the PQAs. E.g. if PQA-2 may be more important than PQA-1, which in turn is more important than PQA-3, then PQA-2 may be assigned a weight indicating that PQA-2 should be prioritized before PQA-1 and PQA-3 when adjusting the process parameters. Furthermore, PQA-1 is assigned a weight indicating that PQA-1 should be prioritized before PQA-3, but not before PQA-2 when adjusting the process parameters.

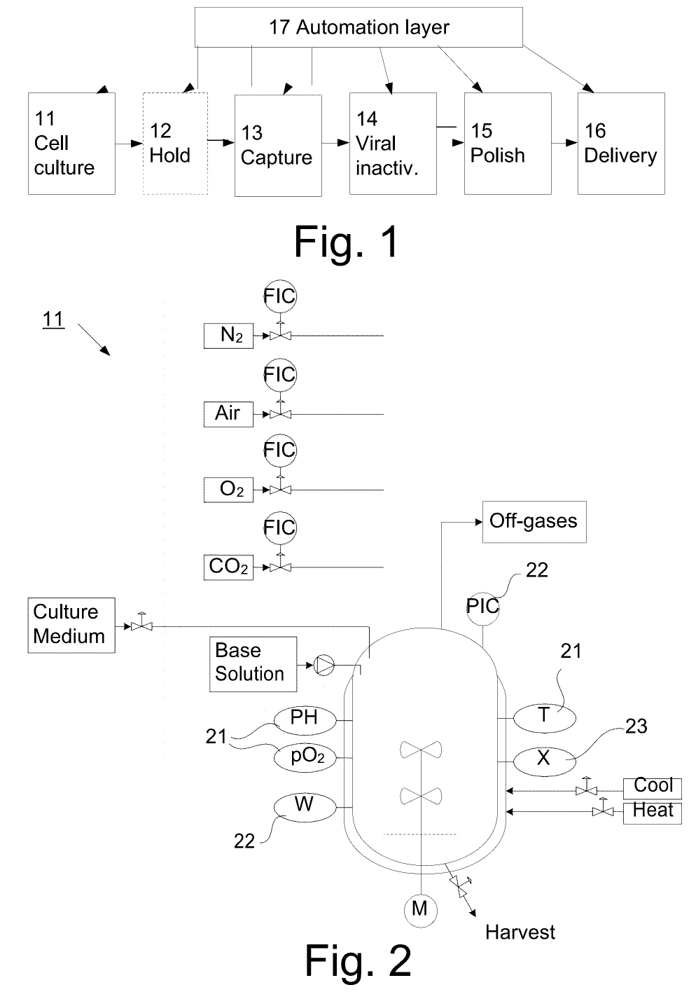

The bioreactor is a part of a bioprocess purification system, and an overview is illustrated in FIG. 1, which is configured to purify a target product using a separation process. The bioprocess purification system comprises a number of steps related to Cell culture 11, Hold 12, Capture 13, Viral inactivation 14, Polish 15 and Delivery 16.

In the disclosed embodiment of the present invention, the cell culture step 11 may be a batch type or continuous cell culture process which comprises of addition of nutrients and removal of product and waste over a limited or an extended period of time (harvest). The cell culture step may comprise process control for viable cell density, VCD, but also nutrients and metabolites. The VCD, productivity and product quality may be controlled by adapting the components of the cell culture media fed to the culture or by addition of certain components directly to the culture.

In some embodiments, the harvest containing the target product may be clarified before feeding the harvest to the downstream purification process, e.g. by filtration, centrifugation or another technique.

The hold step 12 is an optional step depending on process needs, e.g. if a filter is in-line before capture step 13. The step may comprise process control on weight, and the next step in the process starts when a pre-determined volume value is reached, or alternatively after a certain time period or when a pre-determined mass is reached. The hold step may be used for collecting a volume of filtered feed from a perfusion cell culture.

In the disclosed embodiment, the downstream purification process comprises three steps Capture 13, Viral inactivation 14 and Polish 15. The capture step 13 may comprise a chromatography process either in a single chromatography column or a plurality of chromatography columns sequentially connected (as described below). The sequentially connected chromatography involves connecting a first column being supplied by harvest from the cell culture step 11, at a certain point of time disconnecting the first column and connecting a second column in place of the first column, and so on. A filter may be provided in-line before the capture step. The capture step comprises multiple batch elutions, and process control e.g. using in-line UV-sensors handles variation in feed concentration and resin capacity. The next step starts when a pre-determined amount value (e.g. volume, mass or time) is reached.

In the viral inactivation step 14, different options for virus inactivation is available depending on process needs. One option is to use batch mode with low pH for 30-60 minutes in a hold up tank. The step may comprise process control on volume, time, temperature and pH. The next step starts when a pre-determined time is reached.

The polish step 15 may be straight through processing (STP) with a connected batch step or continuous chromatography with a continuous load step, or a combination thereof. The flow rate is adjusted to perfusion rate required by producer cells, which means that the flow rate is determined by the preceding step. The step may comprise process control for UV, flow and volume, and the next step starts when a pre-determined volume and amount is reached, alternatively when a timeout is reached.

The delivery step 16 may comprise a virus removal step, e.g. a viral filter, before an ultra-filtration step. The delivery step may be used as concentration step for batch addition of processed harvest from polish step. The delivery step 16 may comprise continuous or batch delivery of product and may comprise continuous or batch removal of waste. The step may comprise process control for pH, conductivity, absorbance, volume and pressure, and delivery is achieved when a pre-determined product concentration in a pre-defined environment is reached.

An automation layer 17 is used for handling decision points for next step in the process. Different type of sensors (not shown), such as in-line sensors and off-line sensors, are integrated into the process flow to monitor different parameters that may be used for providing the automation layer 17 with data that could be used to handle the decision points. Sensors include but are not limited to only measure flow, VCD, weight, pressure, UV, volume, pH, conductivity, absorbance, nutrients, metabolites, etc.

It should be noted that UV absorption is an example of a parameter that could be monitored to detect the composition of the harvest being purified. However, other parameters may be used operating in other frequency ranges, such as IR, fluorescence etc.

The product quality of the target product produced in a bioprocess purification system may be improved by obtaining information related to the target product during the process run, or the produced target product itself. Attributes relevant to product quality have to be measured, and different analytic methods may be used such as Mass Spectroscopy, MS, Light Scattering, Size Exclusion Chrom, SEC, Raman spectroscopy, etc.

FIG. 2 discloses a cell culture system 11 comprising a bioreactor 20 that produces a harvest containing the target product and the cell culture process may be controlled to optimize the product quality of the target product. Examples of parameters that may be controlled in the bioreactor is temperature, aeration, agitation, cultivation time, pH etc.

In-line sensors 21, or probes, for temperature, pH and partial pressure of oxygen (pO2) are normally present on bioreactors for monitoring and control purposes. They may be installed in situ and are in that case steam-sterilizable in place. Alternatively, in single use settings, the sensors are preinstalled in e.g. the cell cultivation bag, and provided pre-sterilized, commonly by gamma irradiation. The sensors are in such a setting providing the same type of information as described above, but are discarded together with the rest of the single use equipment after use.

In-situ sensors are normally covered with a housing that provides pressure balance during sterilization and other pressurization operations, as well as protection from contamination. Additionally, pilot- and production-scale bioreactors are commonly equipped with on-line devices 22 to measure the liquid level in, or weight of, the bioreactor and the pressure in the headspace. The use of an in situ probe 23 for biomass is also common, at least as a complement to off-line analysis. FIG. 2 shows a simplified instrumentation diagram of a pilot- or large-scale bioreactor operated in batch mode, with the standard sensors. Their characteristics are discussed below. Sensors to measure the flow rate of supplied media and gases are not used as independent monitoring devices but are embedded in a combined monitoring and controlling device.

The main control loops (dotted lines) are also shown, in a schematic way. In the drawing, the following acronyms have been used:
C: controller
F: flow rate
I: indicator
M: motor
P: pressure;
T: temperature
X: biomass
W: liquid weight

Thus, FIC indicates a flow rate indicator controller and PIC is a pressure indicator controller.

Different types of measurements may be performed on a cell culture system, and below the different types are defined by American Pharmaceutical Review™ in a document with the title “Process Control and Monitoring for Continuous Production of Biopharmaceuticals”.
In-line measurement: Sensor is directly interfaced to the process solution.
On-line: Measurements are made in a secondary recirculation loop.
At-line measurements: sample is transferred from the process solution to an analytical instrument in close physical proximity to the process and returns analytical result in a short time cycle.
Off-line measurements: Sample is removed from the process solution and analyzed at a different location and does not returns analytical result in a short time cycle.

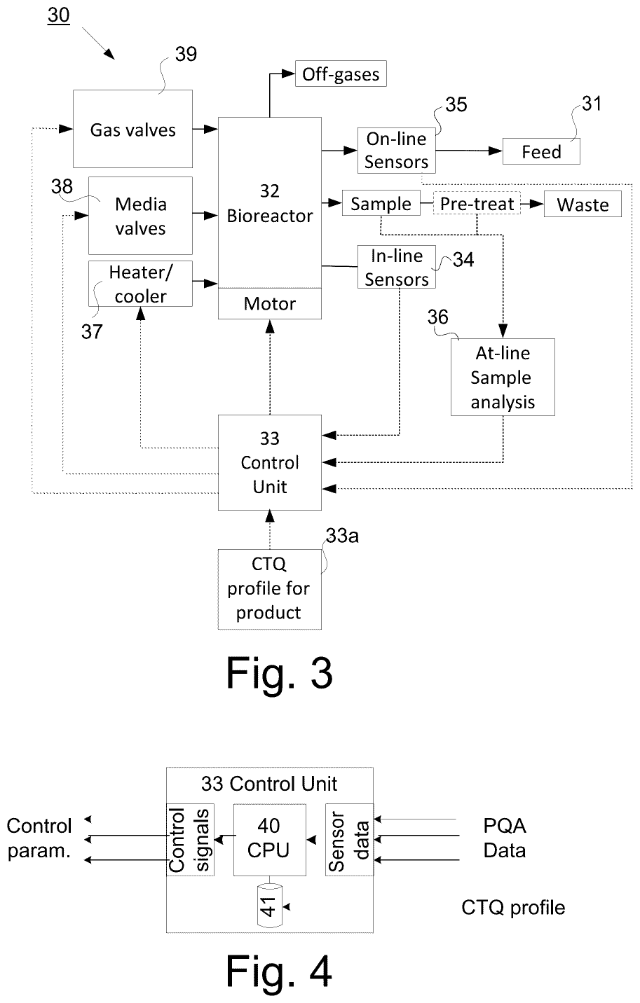

FIG. 3 illustrates an example embodiment of a cell culture system 30 with an improved control system configured to produce a feed 31 (i.e. harvest) containing the target product. The cell culture system 30 comprises a bioreactor 32 provided with a motor controlled by a control unit 33 to provide stirring to the bioreactor, as illustrated in FIG. 2. The cell culture system 30 further comprises a number of sensors 34, 35 configured to provide data to the control unit 33. Some sensors are in-situ sensors 34 monitoring certain parameters within the bioreactor 32 (in-line), such as temperature, DO, pH, etc., and some sensors are on-line sensors 35 monitoring parameters, such as cell related parameters such as viable cell density (VCD) total cell density (TCD), when extracting feed 31 from the bioreactor.

Alternatively, the control unit 33 receives data from an external analyzing equipment 36 requiring that a sample is extracted from the bioreactor 32 (at-line), and optionally pre-treated before sample analysis is performed in the external analyzing equipment 36 and data related to PQA is forwarded to the control unit 33. The control unit 33 process the received data to determine different PQAs that are important for the quality of the target product. As mentioned above, each PQA may be determined based on acquired data from sensors 34, 35 within the cell culture system and/or external analyzing equipment 36.

The control unit 33 is also configured to obtain information regarding the CTQ profile 33a of the target product in order to determine necessary actions to optimize the process parameters to obtain a high quality target product in the feed. These actions may include controlling the temperature of the bioreactor 32 via a heater/cooler 37, adjust culture media introduced into the bioreactor or adjusting the composition of the cell culture media 32 by controlling media valves 38 and/or optimize gas environment 39 within the bioreactor 32 by controlling gas valves.

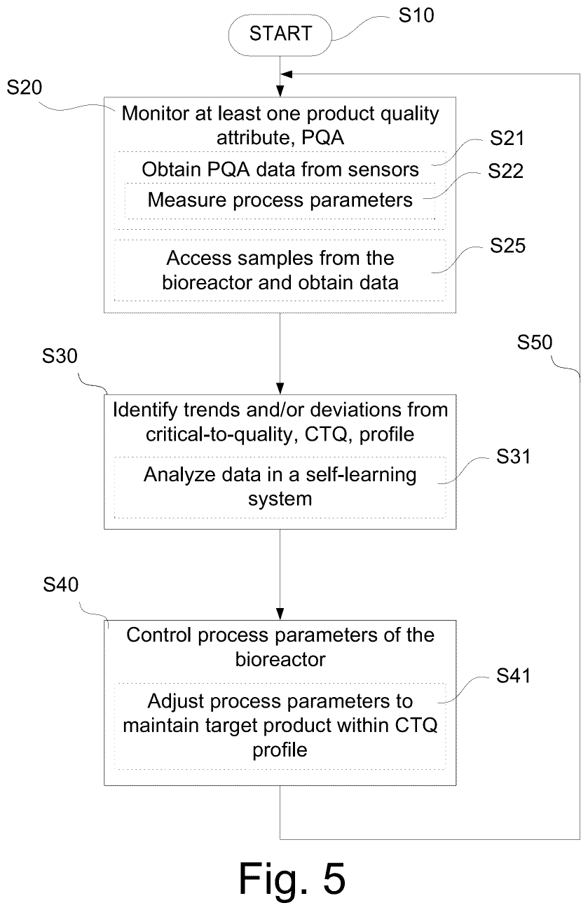

FIG. 4 is an illustrative example of a control unit 33 configured to perform the method as disclosed in connection with FIG. 5. The control unit 33 comprises a central processing unit, CPU, 40 and a memory 41 configured to receive CTQ profile related to the target product under production in the bioreactor. The control unit 33 further receives PQA data (from sensors 34, 35 and/or analyzing equipment 36 of FIG. 3) via an interface 42, and processes the PQA data to determine a value representing the status of at least one PQA at a specific point in time. The value of the at least one PQA is compared to the CTQ profile of the product stored in the memory 41 to identify any trends and/or deviations. The result of the comparison is a decision to send control signals, by controlling process parameters of the bioreactor, to maintain present and future values of the at least one PQA within the CTQ profile of the target product.

FIG. 5 illustrates a method for controlling process parameters in a bioreactor when producing a target product with a pre-determined critical to quality, CTQ, profile. The method starts at S10 and comprises there main steps.

The first step of monitoring S20 at least one product quality attribute, PQA, for the target product. According to some embodiment, the at least one PQA of the target product is selected to be any of polypeptide sequence variations, posttranslational modifications, degradation of the target molecule and/or aggregation.

The second step of identifying S30 trends and/or deviations by comparing the monitored at least one PQA to the CTQ profile of the target product.

The third step of controlling S40 process parameters of the bioreactor based on the identified trends and/or deviations to maintain the target product within the CTQ profile.

According to some embodiment, the step of monitoring S20 the at least one PQA further comprises obtaining S21 data from at least a first sensor system directly mounted in or at the bioreactor to establish the at least a first PQA. The first sensor system includes in-situ sensors and/or in-line sensors, as described in connection with FIG. 3, to provide PQA data from sensors within the cell culture system. According to some embodiments, the PQA data is obtained by measuring S22 selected parameters within the bioreactor. These parameters may include pH, temperature, chemical composition, gas, etc.

According to some embodiments, the step of monitoring S20 the at least one PQA further comprises accessing S25 a sample from the bioreactor and obtaining PQA data from the sample by at least a second sensor system installed at-line or on-line to the bioreactor to establish the at least one PQA. According to some embodiments, accessing S25 a sample includes accessing material from the bioreactor using an automatic sampling system. In some embodiments the step of accessing S25 a sample also includes any pre-treatment of the sample such as filtering, buffer change, or separation/purification of sample components.

According to some embodiments, each of the at least first sensor system and the at least second sensor system is based on spectroscopic methods, surface plasmon resonance, mass spectroscopy, light scattering or light absorbance.

According to some embodiments, the step of monitoring S20 at least one PQA is performed in real-time or near real-time, i.e. at least at regular short time intervals, as illustrated by arrow S50 in FIG. 5. A short time interval may be within 30 minutes.

According to some embodiments, the step of identifying S30 trends and/or deviations further comprises analyzing S31 the at least one PQA in a self-learning system. The self-learning system may be implemented in the control unit 33 described in FIGS. 3 and 4, or as a separate system communicating with the control unit.

According to some embodiment, the step of controlling S40 process parameter further comprises adjusting S41 one or more of the following process parameters in the bioreactor: pH, temperature, stirring rate, dissolved oxygen, partial pressure of carbon dioxide and nutrients. Based on the identified trends and/or deviations in step S30, the relevant process parameters may be adjusted to optimize the CTQ (Critical-to-Quality) profile based on either pre-defined models over an algorithm OR direct control of a pre-defined parameter that influences a specific PQA

The methods described above may be implemented in a computer program for controlling a bioprocess production system. The computer program comprises instructions which, when executed on at least one processor, cause the at least one processor to carry out the method according to the different variations described in connection with FIG. 5. The computer program for controlling the bioprocess purification system may be stored on and carried by a computer readable storage medium.

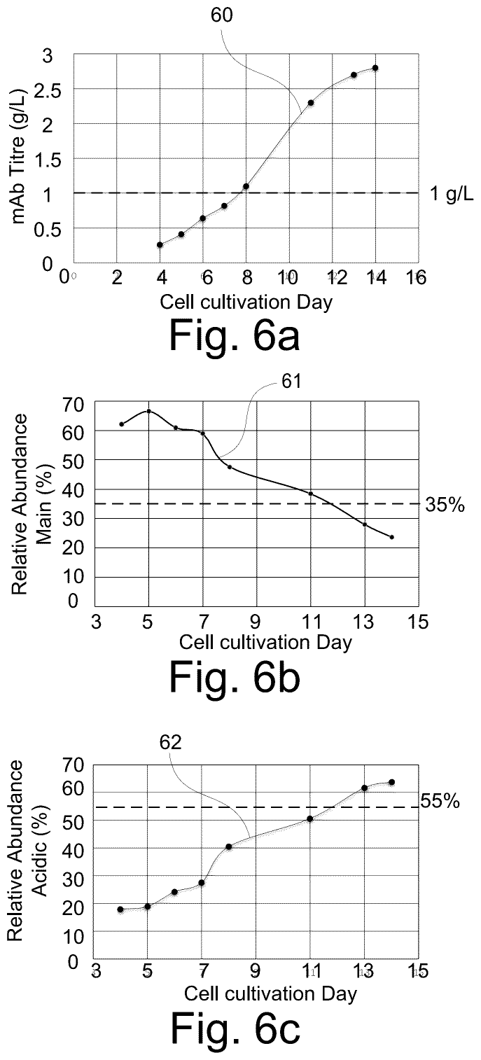

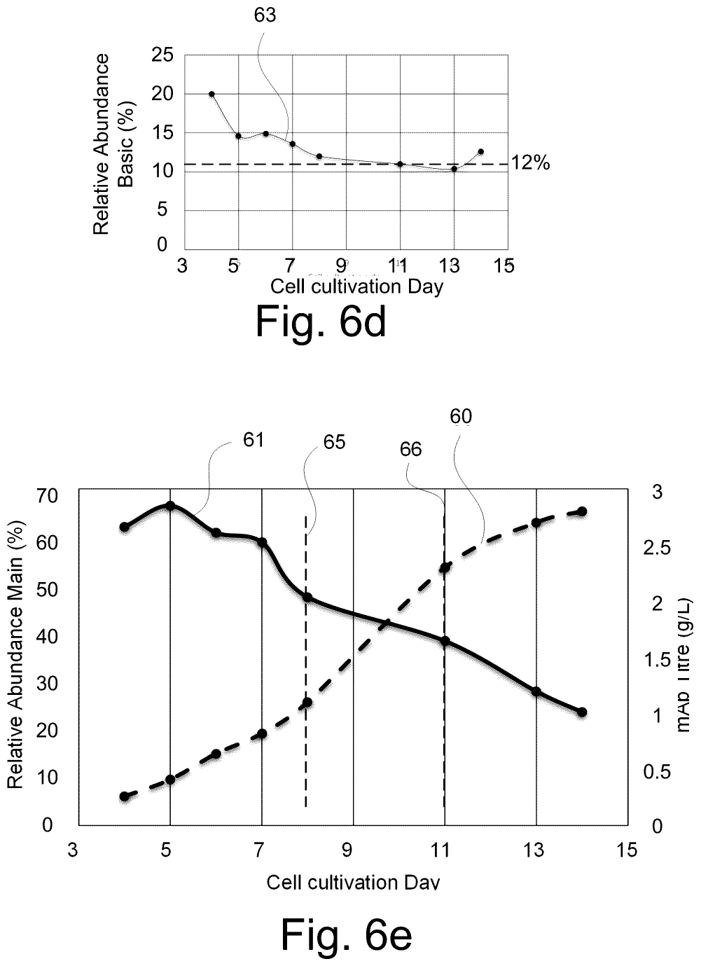

FIGS. 6a-6e illustrate graphs exemplifying the behavior of selected PQAs in a cell culture process without implementing the process described in relation to FIG. 5.

FIG. 6a illustrates mAb concentration, Titre as g/L, in a bioreactor as a function of time (illustrated as cell cultivation day). The concentration is illustrated by a curve 60 corresponding to the concentration of total mAb in the bioreactor. As can be seen from the curve 60, the titre is monitored from day 4 (since it is rather low in the beginning of the cell culture process) and the titre increases to a maximum level at day 14. This is an example of a PQA that is conventionally monitored in bioreactors when producing a target product.

In order for the target product to be suitable for harvest, it is desired that the titre should be above a first threshold. As an example, the first threshold may be defined as when the titre is above 1 g/L, which in this example occurs at cell cultivation day 8.

FIG. 6b illustrates relative abundance of Main mAb species (i.e. the target product as a function of cell cultivation day. Curve 61 indicates that the ratio of target product is largest in the beginning of the process and declines over time.

In order for the target product to be suitable for the following purification process, it is desired that the relative abundance of Main mAb species is above a second threshold. As an example, the second threshold may be defined as when the relative abundance of Main mAb species is below 35%, which in this example means that harvest including cell cultivation day 11 is accepted.

FIG. 6c illustrates relative abundance of Acidic mAb species as a function of cell cultivation day. Curve 62 indicates that the ratio of acidic mAb species is lowest in the beginning of the process and increases over time.

The relative abundance of acidic mAb species should be monitored to avoid having a too high percentage of acidic mAb species when harvesting. As an example, it might be desirable not to harvest when the relative abundance of acidic mAb species is higher than 55%.

FIG. 6d illustrates relative abundance of Basic mAb species as a function of cell cultivation day. Curve 63 indicates that the ratio of basic mAb species is highest in the beginning of the process and decreases over time until it increases in the end of the processing.

FIG. 6e illustrates a combined PQA illustration with the mAb Titre on the right y-axis and the relative abundance of target product on the left y-axis, both as a function of cell cultivation day. A first vertical dashed line 65 at cell culture day “8” represents the point in time from when the Titre is high enough (e.g. over 1 g/L). A second vertical line 66 at cell culture day “11” represents the last point of time when the relative abundance of target product is high enough. At the next cell culture day 12, the relative abundance will be too low (e.g. less than 35%).

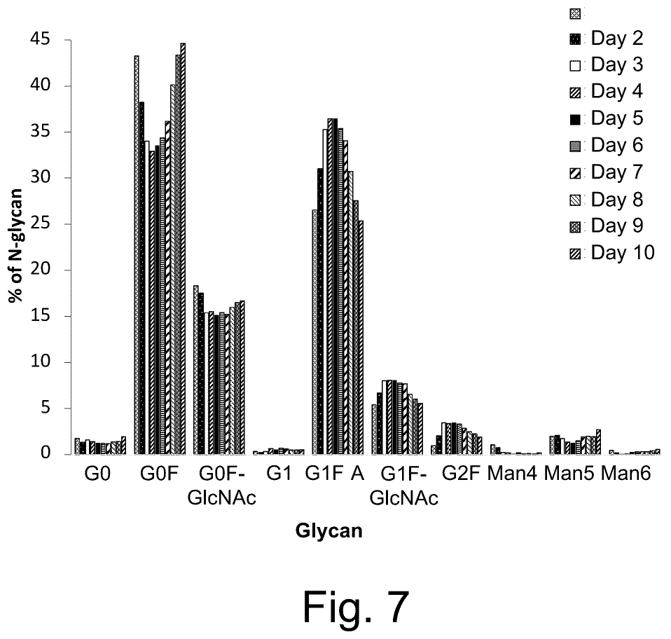

FIG. 7 is a graph illustrating monitored mAb N-glycosylation profile in a bioreactor as a function of cell culture day. The y-axis (% of N-glycan) is normalized in such a way that the sum of all species (G0, G0F, G0F-GlcNAc, G1, G1F A, G1F-GlcNAc, G2F, Man4, Man5 and Man6) is 100%. As illustrated, each species vary over time (From Day 1 to 10).

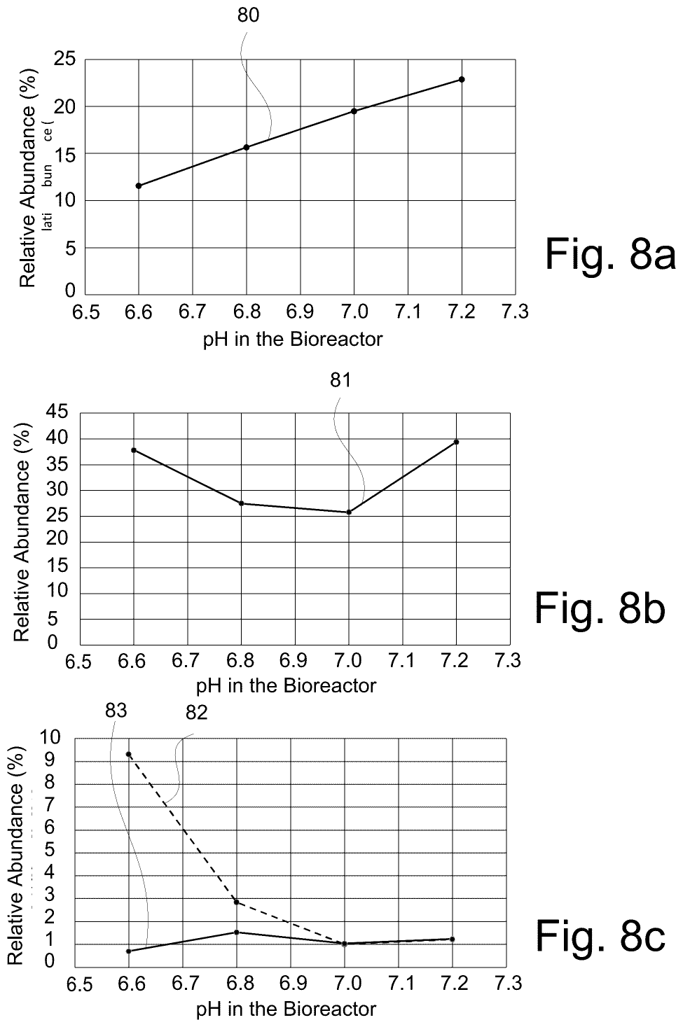

FIGS. 8a-8c illustrate graphs used to control selected PQA as a function of pH in the bioreactor.

In FIG. 8a, curve 80 illustrates that the relative abundance of acidic mAb species in the bioreactor varies linearly as a function of pH. Thus a pH of 6.6 in the bioreactor results in a lower percentage of acidic mAb species than a pH of 7.2 in the bioreactor.

In FIG. 8b, curve 81 illustrates that the relative abundance of basic mAb species in the bioreactor varies as a function of pH. In this case, the relative abundance of basic mAb species is lowest when pH is in the range of 6.8-7.0 in the bioreactor, and the relative abundance of basic mAb species increase when the pH is lower than 6.8, or higher than 7.0, in the bioreactor.

FIG. 8c illustrates the pH effect on mAb aggregate formation. The dashed curve 82 illustrates relative abundance of Aggregates (High Molecular Weight) and the solid curve 83 illustrates relative abundance of Fragments (Low Molecular Weight). Normally, it is desired to have a low relative abundance of Aggregates, which means that a pH higher than 6.8 (or even 7.0) is desired.

As exemplified in FIGS. 8a-8c, pH is only one type of parameter that may be controlled in the bioreactor to keep the selected PQA within the CTQ profile of the target product in order to produce harvest from the bioreactor for a longer time. It is possible to extend the harvest by more days if the relevant parameters are controlled, e.g. by reducing the pH, to reduce the relative abundance of acidic mAb species after day 11, or reduce the amount of relative abundance of aggregates.

1-12. (canceled)

13. A method for controlling process parameters in a bioreactor when producing a target product with a pre-determined critical to quality, CTQ, profile, the method comprising:
monitoring at least one product quality attribute, PQA, for the target product;
identifying trends and/or deviations by comparing the monitored at least one PQA to the CTQ profile of the target product; and
controlling process parameters of the bioreactor based on the identified trends and/or deviations to maintain the target product within the CTQ profile.

14. The method according to claim 13, wherein the step of monitoring the at least one PQA further comprises obtaining PQA data from at least a first sensor system directly mounted to the bioreactor to establish the at least a first PQA.

15. The method according to claim 14, wherein the PQA data is obtained by measuring selected parameters within the bioreactor.

16. The method according to claim 13, wherein the step of monitoring the at least one PQA further comprises accessing a sample from the bioreactor and obtaining data from the sample by at least a second sensor system installed at-line or on-line to the bioreactor to establish the at least one PQA.

17. The method according to claim 14, wherein each of the at least first sensor system and the at least second sensor system is based on spectroscopic methods, surface plasmon resonance, mass spectroscopy, light scattering or light absorbance.

18. The method according to claim 13, wherein the at least one PQA of the target product is selected to be polypeptide sequence variations or posttranslational modifications or degradation of the target molecule.

19. The method according to claim 13, wherein the step of monitoring at least one PQA is performed in real-time or near real-time.

20. The method according to claim 19, wherein the step of identifying trends and/or deviations further comprises analyzing the at least one PQA in a self-learning system.

21. The method according to claim 13 wherein the step of controlling process parameter further comprises adjusting one or more of the following process parameters in the bioreactor: pH, temperature, stirring rate, dissolved oxygen, partial pressure of carbon dioxide and nutrients.

22. A system for producing a feed that includes a target product, said system comprising:
a bioreactor and a control unit configured to control process parameters in the bioreactor according to the method of claim 13.

23. A computer program for controlling process parameters in a bioreactor, comprising instructions which, when executed on at least one processor, cause the at least one processor to carry out the method according to claim 13.

24. A computer-readable storage medium carrying a computer program for controlling process parameters in a bioreactor according to claim 23.
